(19) United States
(12) Patent Application Publication
(10) Pub. No.: US 2023/0142596 A1
(43) Pub. Date: May 11, 2023

(54) IMMUNE RESPONSE PREDICTION FROM SPATIAL TRANSCRIPTOME

(71) Applicant: International Business Machines Corporation, Armonk, NY (US)

(72) Inventors: Ze Fang Tang, Beijing (CN); Xiang Yu Hao, Beijing (CN); Sheng Hu, Beijing (CN); Xiao Yin Zhou, Beijing (CN)

(21) Appl. No.: 17/522,054

(22) Filed: Nov. 9, 2021

(51) Int. Cl. G16H 50/20; G16B 40/10; G16B 15/00; G16B 20/00; G16B 30/00

(52) U.S. Cl. CPC G16H 50/20; G16B 15/00; G16B 20/00; G16B 30/00; G16B 40/10

(57) ABSTRACT

Single cell ribonucleic acid sequencing data has provided numerous avenues to monitor and study organism more thoroughly at a cellular level, including spatial arrangement of cells. An approach to predicting cellular immune response based on cellular spatial features may be presented herein. The approach may include utilizing ribonucleic acid sequence data for a single cell (“scRNA-seq”) or cell from a tissue. The approach may also include extracting spatial features of the single cell using the scRNA-seq data including cell-to-cell interactions and relative distance between cells. The approach may include predicting an immune response of a cell or cells based on the extracted spatial features.

### BACKGROUND OF THE INVENTION

The present invention relates generally to machine learning, more specifically to predicting immune response of a cell based on spatial transcriptome information.

Single cell RNA sequencing (“scRNA-seq”) has led to revolutionary discoveries in researching cancer, diseases, and embryonic development. Many scRNA-seq datasets for various types of tissues have been collected. This trend in collecting scRNA-seq datasets is expected to continue with a tremendous amount of single cell transcriptome data being collected over the coming years. Single cell transcriptome data is the set of all RNA transcripts including coding and non-coding within an individual cell. As RNA is an expression of DNA, specifically mRNA for coding proteins, transcriptome data can provide an overall snapshot into the makeup of the ligand receptors in the cell membrane of a single cell. Ligand-receptors have specific affinities and are typically located near other associated ligand receptors. Cellular organization and interactions can be interpolated from the transcriptome data.

### SUMMARY

Embodiments of the present disclosure include a computer-implemented method, computer program product, and a system for predicting single cell immune response based on single cell ribonuclease sequence data. The embodiments may include receiving a single cell ribonucleic acid sequence (“scRNA-seq”) data. Embodiments may also include extracting spatial features of the single cell, based at least in part on the scRNA-seq data. Additionally, embodiments may include predicting an immune response for the single cell based at least in part on the extracted spatial features.

It should be understood, the above summary is not intended to describe each illustrated embodiment of every implementation of the present disclosure.

### BRIEF DESCRIPTION OF THE DRAWINGS

FIG. 1 is a functional block diagram generally depicting immune response prediction environment 100, in accordance with an embodiment of the present invention.

FIG. 2 is a flowchart depicting operational steps of a method for predicting immune response from single cell ribonuclease data, in accordance with an embodiment of the present invention.

FIG. 3 is a functional block diagram of an exemplary computing system within immune response prediction environment 100, in accordance with an embodiment of the present invention.

FIG. 4 is a diagram depicting a cloud computing environment, in accordance with an embodiment of the present invention.

FIG. 5 is a functional block diagram depicting abstraction model layers, in accordance with an embodiment of the present invention.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

The embodiments depicted and described herein recognize the benefits of utilizing cellular spatial features to predict immune response of a cell.

Single-cell ribonucleic acid sequencing (“scRNA-seq”) has emerged as a revolutionary approach to dissecting cellular compositions and characterizing molecular properties of complex tissues. It should be noted throughout this description, RNA sequence data for a single cell or population of cells will be referred to as a transcriptome, and the two will be used interchangeably. A transcriptome is the set and concentration of all RNA transcripts including coding and non-coding within a cell or populations of cells. Transcriptome data can assist with establishing the protein expression within a cell or population of cells and reveal phylogenic details about the cell or population of cells. Transcriptome data can be used to generate the spatial features of the single cell.

In an embodiment of the present invention, the single cell transcriptome data can be received. The single cell transcriptome data can include all of the coding and non-coding messenger ribonucleic acid (“mRNA”) of the cell. Spatial information for the cell can be derived from the transcriptome data. The spatial data can include cell relative distance and cell affinity or the cell’s expected interaction (based on the known ligand-receptors of the cell). The spatial data can be utilized to predict the immune response of the cell. For example, the immune response can be to a cancer treatment or similar pharmacologic agent.

In describing embodiments in detail with reference to the figures, it should be noted that references in the specification to “an embodiment,” “other embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, describing a particular feature, structure, or characteristic in connection with an embodiment, one skilled in the art has the knowledge to affect such feature, structure or characteristic in connection with other embodiments whether or not explicitly described.

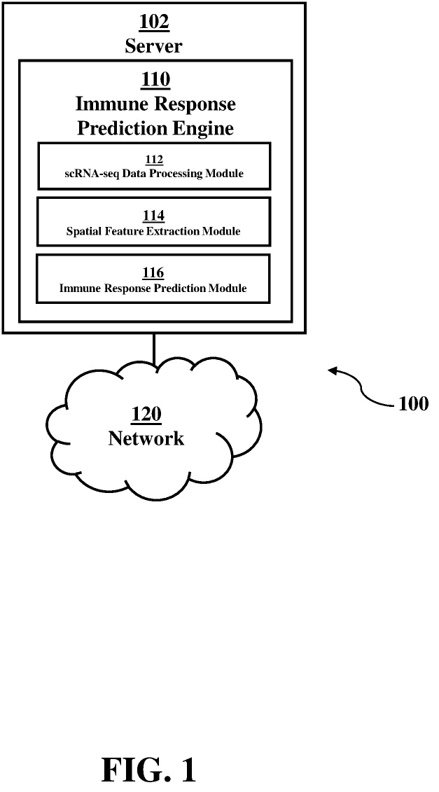

FIG. 1 is a functional block diagram depicting, generally, immune response prediction environment 100. Shown in immune response prediction environment 100 is server 102 and network 120. Also shown in FIG. 1 is immune response prediction engine 110. Operational on immune response prediction engine 110 is scRNA-seq data processing module 112, spatial feature extraction module 114 and immune response prediction module 116.

Server 102 can be a standalone computing device, a management server, a web server, a mobile computing device, or any other electronic device or computing system capable of receiving, sending, and processing data. In other embodiments, server 102 can represent a server computing system utilizing multiple computers as a server system. It should be noted, while one server and one client computer are shown in FIG. 1, cloud resource pre-allocation environment 100 can have any number of servers and client computers (e.g., 1, 2, n...n+1). In another embodiment, server 102 can be a laptop computer, a tablet computer, a netbook computer, a personal computer, a desktop computer, or any programmable electronic device capable of communicating with other computing devices (not shown) within immune response prediction environment 100 via network 120.

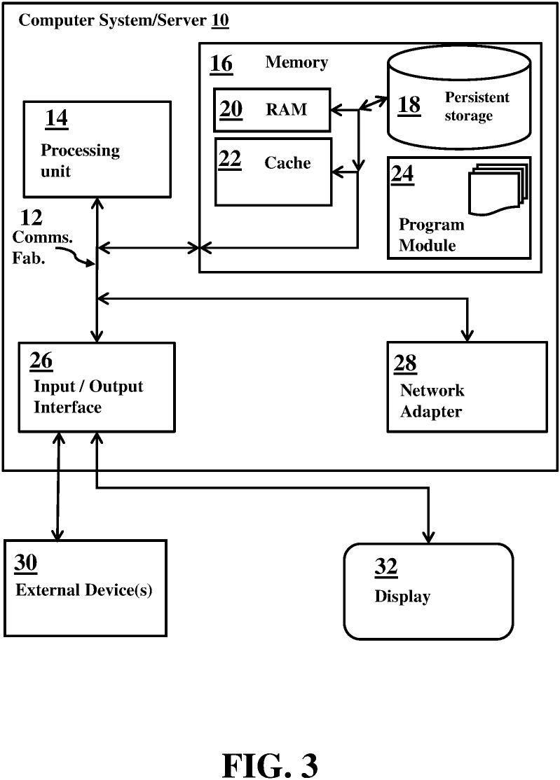

In another embodiment, server 102 represents a computing system utilizing clustered computers and components (e.g., database server computers, application server computers, etc.) that can act as a single pool of seamless resources when accessed within cloud resource pre-allocation environment 100. Server 102 can include internal and external hardware components, as depicted, and described in further detail with respect to FIG. 3.

Operational on server 102 is immune response prediction engine 110. Immune response prediction engine 110 is a computer program that can be configured to utilize scRNA-sequence data to extract spatial features of the single cell and predict an immune response from the extracted spatial features. Immune response prediction engine 110 can be comprised of scRNA-seq data processing module 112, spatial feature extraction module 114 and immune response prediction module 116.

Single cell ribonucleic acid sequence (“scRNA-seq”) data processing module 112 is a computer module that can be configured to receive single cell and process the scRNA-seq data. In an embodiment, scRNA-seq data processing module 112 can receive a dataset of scRNA sequences for one or more single cell types. scRNA-seq data processing module 112 can process the received scRNA-seq data for outlier data. For example, within the dataset of scRNA sequences, the mean concentration of each type of RNA transcripts can be determined for the single cell type. ScRNA-seq data processing module 112 can remove single cell RNA transcripts concentrations samples from the dataset if they fall outside a specific threshold. The threshold can be static or dynamic. It should be noted a threshold can be one or more standard deviations from the calculated mean concentration.

In another embodiment, scRNA-seq data processing module 112 can normalize the transcriptome data of a dataset. For example, scRNA-seq data processing module 112 can receive the single cell transcriptome data for tissue of organ X of multiple donors. The single cell types within the tissue may include immune cells (e.g., T-cells), endothelial cells, epithelium cells, nervous cells, etc.... The RNA transcripts of each cell type can be analyzed and the concentration of each type of RNA transcript can be cataloged. The concentration or number of RNA transcripts can be normalized. For example, scRNA-seq data processing module 112 may perform Z normalization, Min/Max normalization, or unit vector normalization.

In an embodiment, scRNA-seq data processing module 112 can preprocess scRNA-seq data for input into an autoencoder. For example, scRNA-seq can receive a dataset of T-cell scRNA-seq. Data processing module 112 can analyze all of the data with a first pass calculating the mean expression of each mRNA within the scRNA-seq data. Concentrations of non-coding mRNA and coding mRNA that do not contribute to spatial features can be removed by scRNA-seq data processing module 112. The concentration of remaining mRNA can be normalized to a number between 0 and 1, with 1 being the second standard deviation. In other words, keeping the distribution the same, but removing all concentrations outside of the second standard deviation.

Spatial feature extraction module 114 is a computer module that can extract spatial features based on scRNA-seq. For example, spatial feature extraction module 114 can receive processed scRNA-seq of a single cell from scRNA-seq data processing module 112 and extract one or more features of the single cell based on the scRNA-seq. The scRNA-seq can be from a cancer cell in which a specific ligand receptor is over expressed. The over expression would be identified within the transcriptome of the single cell. Spatial feature extraction module 114 may identify features (i.e., vectors) that correspond to an over expression of the ligand receptor. Further, based on the vector, spatial feature extraction module 114 can calculate the cell affinity of the single cell and the relative distance to like type cells and additional cells with the corresponding ligand to the ligand receptor.

In an embodiment, spatial feature extraction module 114 may consist of a model that can receive scRNA-seq data and generate a cell-by-cell affinity matrix for a dataset. A cell-by-cell affinity matrix is a matrix that provides how attracted a cell is to every other cell within the dataset, based on the transcriptome of the cell. For example, spatial feature extraction module 114 may receive a transcriptome dataset of gene expression profiles for one or more cell types within a tissue. The transcriptome dataset can be normalized and/or processed by scRNA-seq data processing module 112. Further, a known ligand receptor network can be fed into spatial feature extraction module 114. The known ligand receptor network can be from a known database (e.g., celltalkDB).

In an embodiment, spatial feature extraction module 114 can embed a cell-by-cell affinity network into a 3-D model. For example, the cell-by-cell affinity network provides the affinity of each cell within a dataset or tissue sample to every other cell within the dataset or tissue sample. Spatial feature extraction module 114 can generate the 3-D model through a nonlinear dimensional reduction algorithm (e.g., t-distributed stochastic neighbor embedding, gaussian process latent variable model, etc.) to embed the cell-by-cell matrix into a 3-D model that shows the density of cell types and cell clusters in real space.

In an embodiment, spatial feature extraction module 114 can generate the ligand-receptor significance of a cell cluster. For example, once a cell cluster has been identified, the number of ligand receptors can be identified and the relative distance of the cells/cell cluster to other cell clusters. The number of ligand receptors for a cell contributes to the relative distance. Thus, based on knowledge of the molecular structure of the ligand receptor complex and the number of ligand receptors of a cell, spatial feature extraction module 114 can calculate the significance or contribution each ligand receptor plays in the spatial arrangement or cellular relative distance.

Immune response prediction engine 116 is a computer module that can predict the immune response of one or more cells based on spatial data derived from the cell’s transcriptome. In an embodiment, immune response prediction engine 116 can be a model trained with one or more datasets, where the dataset is comprised of transcriptome data of cells before an immune response (i.e., condition 0) and after an immune response (i.e., condition 1) For example, a dataset may contain a dataset of scRNA-seq for patients with lung cancer. The dataset can have scRNA-seq for cell types of patients before (condition 0) and after treatment with a specific treatment (condition 1), indicating the immune response for the treatment. The spatial features (e.g., cell affinity, cell relative distance, cell to cell interaction) for each sample within the dataset can be extracted for condition 0 and condition 1.

In an embodiment, immune response prediction engine 116 can have an encoding model, which can be fed the spatial features to generate vector encodings for each scRNA-seq. The model can be trained with a dataset of condition 0 and condition 1 scRNA-seq samples. The encoder can further have a decoder, in which the decoder can be used to ensure the latent space of the encoder is accurately encoding the spatial features. Immune response prediction engine 116 can calculate the mean change of all the samples within the latent space from condition 0 to condition 1. Further, immune response prediction engine 116 can receive a scRNA-seq sample for condition 0 and predict condition 1. The prediction is based off an extrapolation of the latent space with the addition of the mean for condition 1. The extrapolated latent space can be fed into the decoder to generate spatial features of the cell in condition 1.

Network 120 can be, for example, a local area network (LAN), a wide area network (WAN) such as the Internet, or a combination of the two, and can include wired, wireless, or fiber optic connections. In general, network 120 can be any combination of connections and protocols that will support communications between server 102, and other computing devices within immune response prediction environment 100.

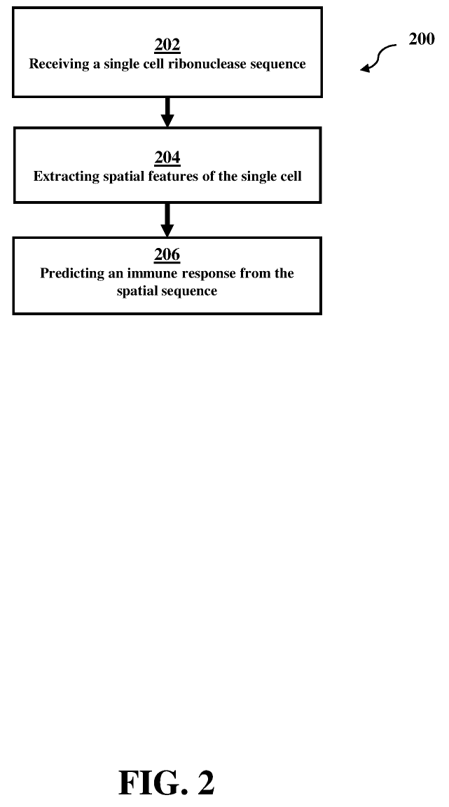

FIG. 2 is a flowchart, generally designated 200, depicting operational steps of predicting immune response based on spatial transcriptome information.

At step 202, scRNA-seq data processing module receives scRNA-seq data. For example, multiple samples of a specific tissue or cell type can be received at scRNA-seq data processing module 112. The scRNA-seq data of the dataset may be processed (e.g., removing outlier data, non-coding data, and/or normalized). In another example, scRNA-seq data processing module 112 can receive a scRNA-seq data for a single cell. The concentrations of mRNA that do not contribute to cellular spatial organization can be removed by scRNA-seq data processing module 112.

At step 204, spatial feature extraction module 114 can receive or obtain the processed scRNA-seq data from scRNA-seq data processing module 112 and extract spatial features from the processed scRNA-seq data. For example, spatial feature extraction module 114 can generate a cell-by-cell affinity matrix based on the processed scRNA-seq data of a dataset. In another example, the scRNA-seq data of a single cell type can be received by scRNA-seq data processing module 112 to extract the cell relative distance of the cells. In yet another example, spatial feature extraction module 114 can identify the number and type of ligand receptors in a scRNA-seq dataset and determine the cell-to-cell interactions of a dataset based on the significance of the ligand receptors affinity to nearby cells and cell types.

At step 206, immune response prediction module 116 can predict an immune response based on the spatial features extracted by spatial feature extraction module 114. For example, immune response prediction module 116 can receive the extracted spatial features of a single cell from the cell’s scRNA-seq in condition 0. Immune response prediction module 116 can be trained to predict the spatial features of one or more cell perturbations (e.g., developmental or age related issue) or treatments (e.g., a cancer drug or antiviral medication). The extracted spatial features can be fed into an encoding model. Based on the vectors that are encoded, immune response prediction module 116 can extrapolate the encodings to condition 1 based on previously obtained mean difference between the encodings of cells in condition 0 and condition 1. The extrapolated encodings can be fed into a decoder to provide spatial features (gene expression, cell-to-cell interaction, cell relative distance, etc.) which correspond to an immune response.

FIG. 3 depicts computer system 10, an example computer system representative of a dynamically switching user interface computer 10. Computer system 10 includes communications fabric 12, which provides communications between computer processor(s) 14, memory 16, persistent storage 18, network adaptor 28, and input/output (I/O) interface(s) 26. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses.

Computer system 10 includes processors 14, cache 22, memory 16, persistent storage 18, network adaptor 28, input/output (I/O) interface(s) 26 and communications fabric 12. Communications fabric 12 provides communications between cache 22, memory 16, persistent storage 18, network adaptor 28, and input/output (I/O) interface(s) 26. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses or a crossbar switch.

Memory 16 and persistent storage 18 are computer readable storage media. In this embodiment, memory 16 includes random access memory (RAM) 20. In general, memory 16 can include any suitable volatile or non-volatile computer readable storage media. Cache 22 is a fast memory that enhances the performance of processors 14 by holding recently accessed data from memory 16, nearby processors 14. As will be further depicted and described below, memory 16 may include at least one of program module 24 that is configured to carry out the functions of embodiments of the invention.

The program/utility, having at least one program module 24, may be stored in memory 16 by way of example, and not limiting, as well as an operating system, one or more application programs, other program modules, and program data. Each of the operating systems, one or more application programs, other program modules, and program data or some combination thereof, may include an implementation of a networking environment. Program module 24 generally carries out the functions and/or methodologies of embodiments of the invention, as described herein.

Program instructions and data used to practice embodiments of the present invention may be stored in persistent storage 18 and in memory 16 for execution by one or more of the respective processors 14 via cache 22. In an embodiment, persistent storage 18 includes a magnetic hard disk drive. Alternatively, or in addition to a magnetic hard disk drive, persistent storage 18 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Network adaptor 28, in these examples, provides for communications with other data processing systems or devices. In these examples, network adaptor 28 includes one or more network interface cards. Network adaptor 28 may provide communications through the use of either or both physical and wireless communications links. Program instructions and data used to practice embodiments of the present invention may be downloaded to persistent storage 18 through network adaptor 28.

I/O interface(s) 26 allows for input and output of data with other devices that may be connected to each computer system. For example, I/O interface 26 may provide a connection to external devices 30 such as a keyboard, keypad, a touch screen, and/or some other suitable input device. External devices 30 can also include portable computer readable storage media such as, for example, thumb drives, portable optical or magnetic disks, and memory cards. Software and data used to practice embodiments of the present invention can be stored on such portable computer readable storage media and can be loaded onto persistent storage 18 via I/O interface(s) 26. I/O interface(s) 26 also connect to display 32.

Display 32 provides a mechanism to display data to a user and may be, for example, a computer monitor or virtual graphical user interface.

The components described herein are identified based upon the application for which they are implemented in a specific embodiment of the invention. However, it should be appreciated that any particular component nomenclature herein is used merely for convenience, and thus the invention should not be limited to use solely in any specific application identified and/or implied by such nomenclature.

The present invention may be a system, a method and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems) and computer program products according to embodiments of the invention. It is understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality and operation of possible implementations of systems, methods, and computer program products according to various embodiments of the present invention. In this regard, each block in the flowchart or block diagrams may represent a module, segment or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service’s provider.

Broad network access: capabilities are available over a network and accessed through standard mechanisms that promote use by heterogeneous thin or thick client platforms (e.g., mobile phones, laptops, and PDAs).

Resource pooling: the provider’s computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider’s applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

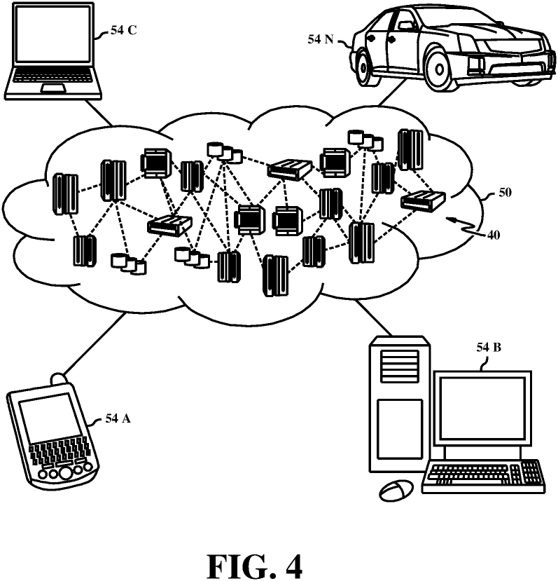

FIG. 4 is a block diagram depicting a cloud computing environment 50 in accordance with at least one embodiment of the present invention. Cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

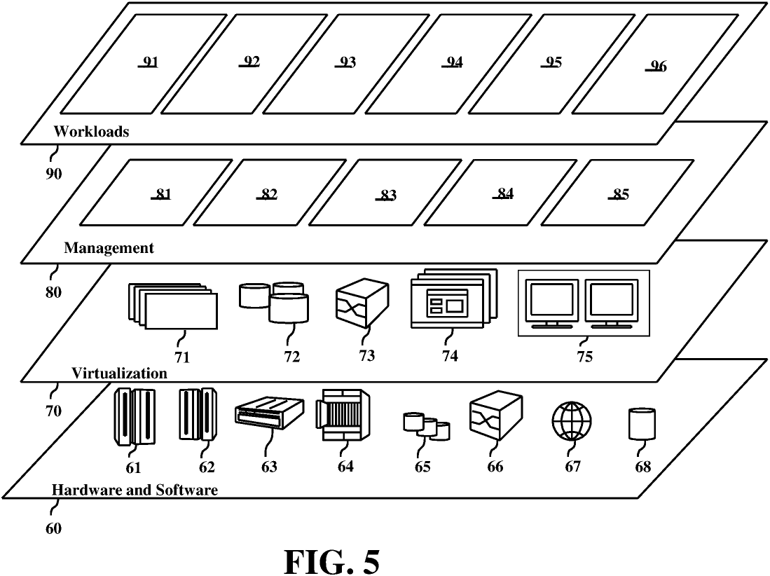

FIG. 5 is a block diagram depicting a set of functional abstraction model layers provided by cloud computing environment 50 depicted in FIG. 4 in accordance with at least one embodiment of the present invention. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and cellular immune response prediction 96.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for predicting a cellular level immune response, the computer-implemented method comprising:
receiving, by a processor, a single cell ribonuclease sequence (“scRNA-seq”);
extracting, by the processor, spatial features of the single cell based, at least in part, on the scRNA-seq; and
predicting, by the processor, an immune response based, at least in part, on the extracted spatial features.

2. The computer-implemented method of claim 1, wherein extracting spatial features of the single cell further comprises:
generating, by the processor, a cell-by-cell affinity matrix based, at least in part, on the scRNA-seq; and
determining, by the processor, a relative distance based, at least in part, on the cell-by-cell affinity matrix.

3. The computer-implemented method of claim 2, wherein determining the relative distance of cell pair clusters further comprises:
generating, by the processor, a three-dimensional model of the cell-by-cell affinity matrix, wherein the three-dimensional model is based on non-linear extrapolation of the cell-by-cell affinity matrix;
determining, by the processor, one or more cell type densities based, at least in part, on the three-dimensional model;
determining, by the processor, one or more cell pair clusters based, at least in part, on the one or more cell type densities;
determining, by the processor, a number of ligand-receptor connections between cell pair cluster interactions based, at least in part on the one or more cell pair clusters and the cell-by-cell affinity matrix; and
calculating, by the processor, a significance of the number of ligand-receptor connections between the one or more cell pair clusters.

4. The computer implemented method of claim 1, further comprising:
receiving, by the processor, a training data set, wherein the training data set is comprised of scRNA-seq data of a plurality of single cell scRNA-seq datasets in a before treatment condition and a corresponding after treatment condition;
encoding, by the processor, the spatial features of each training data set of each single cell type in a latent space; and
calculating, by the processor, the mean difference of the latent space between the before treatment condition and after treatment condition of each corresponding single cells type.

5. The computer implemented method of claim 4, wherein predicting the immune response further comprises:
encoding, by the processor, the latent space, the single cell scRNA-seq, based on the generated cell-by-cell affinity matrix and the determined cell relative distance; and
extrapolating, by the processor, the encoded latent space of the single cell scRNA-seq based on the calculated mean difference between the calculated mean difference between the plurality of single cell scRNA-seq datasets in the before treatment condition and the corresponding after treatment condition.

6. The computer implemented method of claim 1, wherein the spatial features are comprised of a cell-by-cell affinity matrix and a cell relative distance.

7. The computer implemented method of claim 1, wherein the predicted immune response is an immune response to a cancer treatment.

8. A computer system for predicting a cellular level immune response, the system comprising:
one or more computer processors;
one or more computer readable storage media; and
computer program instructions to:
receive a single cell ribonuclease sequence (“scRNA-seq”);
extract spatial features of the single cell based, at least in part, on the scRNA-seq; and
predict an immune response based, at least in part, on the extracted spatial features.

9. The computer system of claim 8, wherein extracting spatial features of the single cell further comprises:
generate a cell-by-cell affinity matrix based, at least in part, on the scRNA-seq; and
determine a relative distance based, at least in part, on the cell-by-cell affinity matrix.

10. The computer system of claim 9, wherein determining the relative distance of cell pair clusters further comprises:
generate a three-dimensional model of the cell-by-cell affinity matrix, wherein the three-dimensional model is based on non-linear extrapolation of the cell-by-cell affinity matrix;
determine one or more cell type densities based, at least in part, on the three-dimensional model;
determine one or more cell pair clusters based, at least in part, on the one or more cell type densities;
determine a number of ligand-receptor connections between cell pair cluster interactions based, at least in part on the one or more cell pair clusters and the cell-by-cell affinity matrix; and
calculate a significance of the number of ligand-receptor connections between the one or more cell pair clusters.

11. The computer system of claim 8, further comprising:
receive a training data set, wherein the training data set is comprised of scRNA-seq data of a plurality of single cell scRNA-seq datasets in a before treatment condition and a corresponding after treatment condition;
encode the spatial features of each training data set of each single cell type in a latent space; and
calculate the mean difference of the latent space between the before treatment condition and after treatment condition of each corresponding single cells type.

12. The computer system of claim 11, wherein predicting the immune response further comprises:
encode the latent space, the single cell scRNA-seq, based on the generated cell-by-cell affinity matrix and the determined cell relative distance; and
extrapolate the encoded latent space of the single cell scRNA-seq based on the calculated mean difference between the calculated mean difference between the plurality of single cell scRNA-seq datasets in the before treatment condition and the corresponding after treatment condition.

13. The computer system of claim 8, wherein the spatial features are comprised of a cell-by-cell affinity matrix and a cell relative distance.

14. The computer system of claim 8, wherein the predicted immune response is an immune response to a cancer treatment.

15. A computer program product for predicting a cellular level immune response, the computer program product comprising one or more computer readable storage device and program instructions sorted on the one or more computer readable storage device to:
receive a single cell ribonuclease sequence (“scRNA-seq”);
extract spatial features of the single cell based, at least in part, on the scRNA-seq; and
predict an immune response based, at least in part, on the extracted spatial features.

16. The computer program product of claim 15, wherein extracting spatial features of the single cell further comprises:
generate a cell-by-cell affinity matrix based, at least in part, on the scRNA-seq; and
determine a relative distance based, at least in part, on the cell-by-cell affinity matrix.

17. The computer program product of claim 16, wherein determining the relative distance of cell pair clusters further comprises:
generate a three-dimensional model of the cell-by-cell affinity matrix, wherein the three-dimensional model is based on non-linear extrapolation of the cell-by-cell affinity matrix;
determine one or more cell type densities based, at least in part, on the three-dimensional model;
determine one or more cell pair clusters based, at least in part, on the one or more cell type densities;
determine a number of ligand-receptor connections between cell pair cluster interactions based, at least in part on the one or more cell pair clusters and the cell-by-cell affinity matrix; and
calculate a significance of the number of ligand-receptor connections between the one or more cell pair clusters.

18. The computer program product of claim 15, further comprising:
receive a training data set, wherein the training data set is comprised of scRNA-seq data of a plurality of single cell scRNA-seq datasets in a before treatment condition and a corresponding after treatment condition;
encode the spatial features of each training data set of each single cell type in a latent space; and
calculate the mean difference of the latent space between the before treatment condition and after treatment condition of each corresponding single cells type.

19. The computer program product of claim 18, wherein predicting the immune response further comprises:
encode the latent space, the single cell scRNA-seq, based on the generated cell-by-cell affinity matrix and the determined cell relative distance; and
extrapolate the encoded latent space of the single cell scRNA-seq based on the calculated mean difference between the calculated mean difference between the plurality of single cell scRNA-seq datasets in the before treatment condition and the corresponding after treatment condition.

20. The computer program product of claim 15, wherein the spatial features are comprised of a cell-by-cell affinity matrix and a cell relative distance.
